(19) United States
(12) Patent Application Publication
(10) Pub. No.: US 2010/0137966 A1
(43) Pub. Date: Jun. 3, 2010

(54) SYSTEM AND METHOD FOR SEQUENTIALLY DEPLOYING TWO OR MORE IMPLANTABLE MEDICAL DEVICES

(75) Inventor: Mark A. Magnuson, Bloomington, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 12/325,511

(22) Filed: Dec. 1, 2008

(51) Int. Cl. A61F 2/06; A61B 19/00

(52) U.S. Cl. 623/1.11; 128/898

(57) ABSTRACT

An intraluminal delivery system for sequentially deploying two or more implantable medical devices includes two or more such devices arranged longitudinally adjacent to each other about an inner catheter. One or more separator bands are slideably disposed about the inner catheter, and each of the bands is positioned between adjacent medical devices. An outer catheter, which is proximal to the medical devices, overlies the inner catheter but does not overlie the medical devices. A tubular sheath overlies the outer catheter, the one or more separator bands, and the medical devices. Relative motion between the tubular sheath and the inner catheter allows the medical devices to be sequentially deployed at one or more treatment sites, and relative motion between the inner catheter and the outer catheter allows a distal tip of the inner catheter to be retracted after deployment of each device.

### TECHNICAL FIELD

The present disclosure is directed generally to medical device delivery systems and, more particularly, to a system and method for sequentially deploying two or more implantable medical devices in one or more body vessels.

### BACKGROUND

Stents are tubular support structures that may be implanted into body vessels to treat blockages, occlusions, narrowing ailments and other problems that can restrict flow through the vessel. Generally, a stent includes a framework of interconnected struts that allows the stent to be collapsed into a low profile configuration for delivery into the vessel and then radially expanded at the treatment site to support the vessel wall. Balloon-expandable stents expand in response to the inflation of a balloon, whereas self-expanding stents deploy automatically when released from a delivery device.

Numerous vessels throughout the vascular system, including peripheral arteries, such as the carotid, brachial, renal, iliac and femoral arteries, and other vessels, may benefit from treatment by a stent. For example, the superficial femoral artery (SFA) may be a site of occlusions or blockages caused by peripheral artery disease. This condition causes leg pain and gangrene in severe cases and affects roughly 8 million to 12 million Americans, according to the American Heart Association.

In some patients, the SFA may include multiple blockages. To treat the patient in such situations, a physician may need to carry out multiple intraluminal procedures to deploy two or more stents in the SFA. In each procedure, a delivery system may be inserted into the SFA carrying a self-expanding stent for the treatment of one of the occluded areas. After the first stent is deployed, the first delivery system can be removed and a second delivery system carrying a second stent for treatment of another occluded area may be inserted into the vessel. Depending on the number of blockages, additional delivery systems may have to be inserted into the SFA for the deployment of additional stents.

The inventor believes an improved method of delivering and deploying multiple stents into the SFA and other body vessels is needed.

### BRIEF SUMMARY

An improved intraluminal delivery system and a method for sequentially deploying two or more implantable medical devices are described. The system and method permit two or more medical devices to be deployed at multiple locations in one or more body vessels in a single procedure. The first device may be deployed at a treatment site that is distal to, proximal to, or the same as that of a succeeding device. The system includes a retractable distal tip that allows the delivery system to be made more compact in vivo as the medical devices are deployed. Accordingly, the system and method may provide increased flexibility in the placement of the implantable medical devices while reducing the likelihood of vascular or organ damage downstream of the treatment sites. The system is also designed to minimize contact between and potential damage to adjacent medical devices during deployment.

The method includes providing an intraluminal delivery system having a delivery configuration comprising two or more implantable medical devices arranged longitudinally adjacent to each other about an inner catheter. One or more separator bands are slideably disposed about the inner catheter, and each separator band is positioned between adjacent medical devices. An outer catheter overlies the inner catheter and is disposed proximal to the medical devices. The outer catheter has an inner diameter large enough to allow for longitudinal motion of the inner catheter but small enough to prevent the medical devices from passing therethrough. The method further includes advancing the intraluminal delivery system to a first treatment site in a body vessel, deploying a first medical device at the first treatment site, and deploying a second medical device at a second treatment site. The inner catheter is moved relative to the outer catheter to retract a distal tip of the inner catheter after deploying at least one of the first and second medical devices. The intraluminal delivery system is removed from the body vessel, and the first and second medical devices remain deployed.

The intraluminal delivery system includes two or more implantable medical devices arranged longitudinally adjacent to each other about an inner catheter. One or more separator bands are slideably disposed about the inner catheter, and each separator band is positioned between longitudinally adjacent medical devices. An outer catheter, which is proximal to the medical devices, overlies the inner catheter but does not overlie the medical devices. A tubular sheath overlies the outer catheter, the bands, and the medical devices. Relative motion between the tubular sheath and the inner catheter allows the medical devices to be sequentially deployed at one or more treatment sites, and relative motion between the inner catheter and the outer catheter allows a distal tip of the inner catheter to be retracted after deployment of each device.

### BRIEF DESCRIPTION OF THE DRAWINGS

FIG. 1 shows a longitudinal cross-sectional view of one embodiment of an intraluminal delivery system for the delivery and deployment of first and second implantable medical devices, where the intraluminal delivery system is in a delivery configuration;

FIG. 2 shows the intraluminal delivery system of FIG. 1 in a partially deployed configuration in which the first medical device is deployed;

FIG. 3 shows the intraluminal delivery system of FIG. 1 in a partially deployed configuration in which the first medical device is deployed and a distal tip of the delivery system is retracted;

FIG. 4 shows the intraluminal delivery system of FIG. 1 in a fully deployed configuration in which the first and second medical devices are deployed;

FIG. 5 shows the intraluminal delivery system of FIG. 1 in a fully deployed configuration with the distal tip further retracted;

FIG. 6 shows a longitudinal cross-sectional view of one embodiment of an intraluminal delivery system for the deployment of three implantable medical devices, where the intraluminal delivery system is in a delivery configuration;

FIGS. 7 to 11 show steps in a method of sequentially deploying two self-expanding stents at first and second treatment sites in a body vessel;

FIG. 12 shows a transverse cross-sectional view of one embodiment of a radiopaque separator or stopper band;

FIG. 13 shows a transverse cross-sectional view of another embodiment of a radiopaque separator or stopper band; and

FIG. 14 shows a transverse cross-sectional view of another embodiment of a radiopaque separator or stopper band.

### DETAILED DESCRIPTION

Throughout the specification, the term “distal” refers to the end of an intraluminal device or component or to part of a body vessel that is farther away from the clinician carrying out the intraluminal procedure, and the term “proximal” refers to the end of an intraluminal device or component or to part of the body vessel that is closer to the clinician carrying out the procedure.

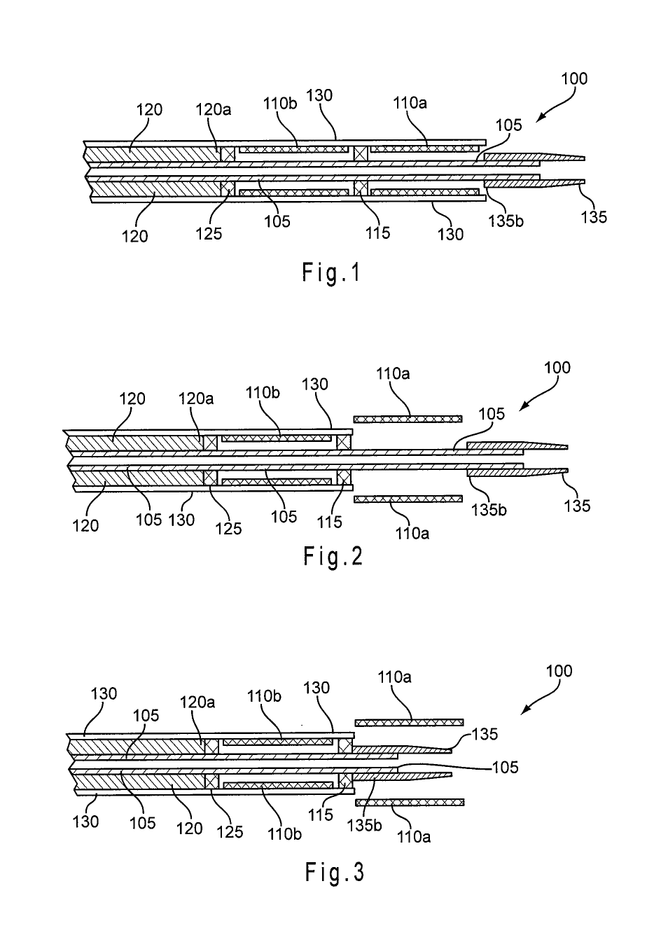

FIG. 1 shows an exemplary intraluminal delivery system for the delivery and deployment of two implantable medical devices in one or more body vessels. The delivery system 100, which is shown in a delivery configuration in FIG. 1, includes an inner catheter 105 underlying a first stent 110a and a second stent 110b and a separator band 115 positioned between the two longitudinally adjacent stents 110a, 110b. The separator band 115 is not fixed in place but rather is slideably disposed about the inner catheter 105. The separator band 115 can thus be rotated about and moved longitudinally along the inner catheter 105. The separator band 115 helps to minimize loading of the second stent 110b by the more distally positioned first stent 110a and to keep the stents 110a, 110b separated. It may be formed in whole or in part of a radiopaque material. The separator band 115 may thus serve as a radiopaque marker which is visible under x-ray irradiation and useful for properly positioning the first and second stents 110a, 110b during delivery and deployment.

An outer catheter 120 overlies the inner catheter 105 and is positioned proximal to the first and second stents 110a, 110b. The outer catheter 120 does not overlie the stents 110a, 110b. The outer and inner catheters 120, 105 are coaxial and moveable relative to each other.

A stopper band 125, which is preferably formed in whole or in part of a radiopaque material, may be positioned at a distal end 120a of the outer catheter 120. The stopper band 125 is slideably disposed about the inner catheter 105. Like the outer catheter 120, the stopper band 125 is proximal to the two stents 110a, 110b. The stopper band 125 may be secured to or integrally formed with the distal end 120a of the outer catheter 120. In some embodiments of the delivery system 100, the stopper band 125 may not be used.

One or both of the outer catheter 120 and the stopper band 125 are sized to contact a proximal end of the second stent 110b. That is, an inner diameter of at least one of the outer catheter 120 and the stopper band 125 is sized to prevent the stents 110a, 110b from passing therethrough, and an outer diameter of at least one of the outer catheter 120 and the stopper band 125 is sized to prevent the stents 110a, 110b from passing thereover in the delivery system 100. The stopper band 125 and/or the outer catheter 120 act as a proximal restraint to the stents 110a, 110b or other medical devices disposed about the inner catheter 105 during delivery and deployment.

The inner catheter 105 may include a tapered distal tip 135 to facilitate smooth travel of the delivery system 100 through the vessel. The tip 135 is generally a separate component attached to the inner catheter 105 by a bonding process, although it is also contemplated that the tip 135 may be integrally formed with the inner catheter 105. In either case, the proximal end 135b of the tip 135 preferably extends radially outward from the inner catheter 105, creating a ledge that may help to retain the medical devices 110a, 110b and separator band(s) 115 in position along the inner catheter 105.

In the delivery configuration, a tubular sheath 130 may overlie the outer catheter 120, the bands 115, 125 and the stents 110a, 110b, leaving the distal tip 135 exposed.

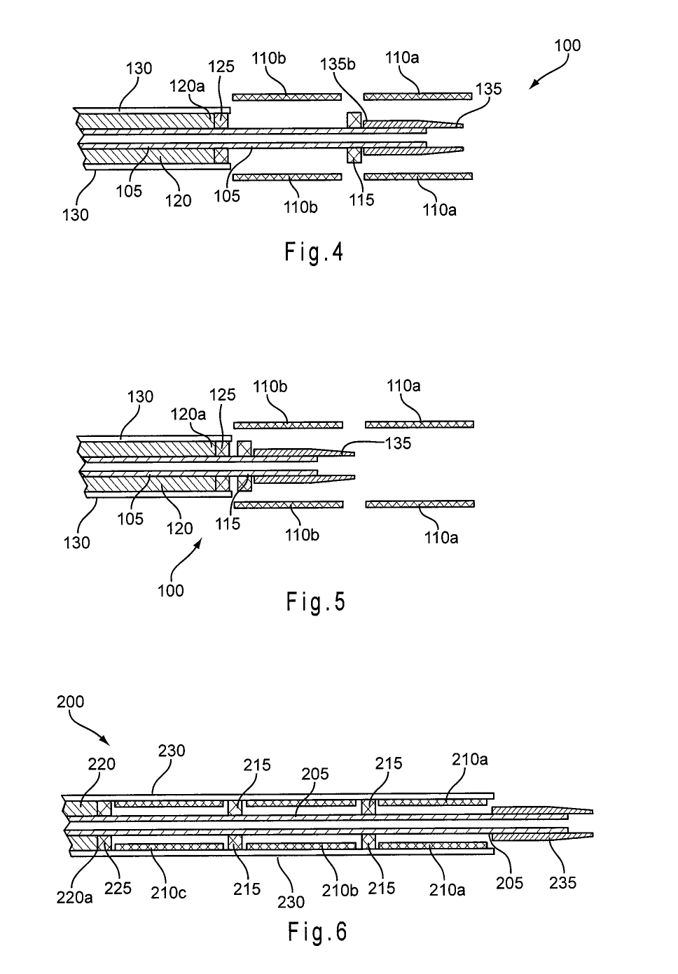

Referring to FIGS. 2-5, relative motion between the tubular sheath 130 and the inner catheter 105 allows the self-expanding stents 110a, 110b to be sequentially deployed at one or more treatment sites. Generally, the sheath 130 is retracted (i.e., moved in a proximal direction) after the inner catheter 105 has been positioned at a desired site in the vessel to deploy the first stent 110a. As the self-expanding stent 110a is freed from the radial constraint provided by the sheath 130, it expands to contact the vessel wall. FIG. 2 shows the first stent 110a in a deployed configuration following partial retraction of the sheath 130. During deployment, the separator band 115 prevents contact between adjacent stents, and the stopper band 125 and/or outer catheter 120 minimize proximal motion of the undeployed stent 110b. Once the first stent 110a has been deployed, the inner catheter 105 may be retracted to position the distal tip 135 immediately adjacent to the separator band 115, as shown in FIG. 3. The distal tip 135 may underlie the deployed device in this position. Upon further retraction of the sheath 130, as shown in FIG. 4, the second stent 110b may be deployed. The deployment of the second stent 110b need not take place at or near the treatment site at which the first stent 110a is placed, as will be discussed further below. FIG. 5 shows retraction of the distal tip 135 following deployment of the second stent 110b.

The preceding paragraphs and FIGS. 1-5 describe an exemplary delivery system configured for the deployment of two longitudinally adjacent medical devices. The system may also be sized to accommodate more than two medical devices.

Referring to FIG. 6, for example, the system 200 may be configured to sequentially deploy three longitudinally adjacent medical devices (e.g., self-expanding stents 210a, 210b, 210c). According to this embodiment, the system includes a separator band 215 between each adjacent device, for a total of two separator bands 215. The system 200 also includes an outer catheter 220 overlying an inner catheter 205 and disposed proximal to the stents 210a-c, as in the previous embodiment. The outer catheter 220 does not overlie the stents 210a-c and is preferably sized so that the stents 210a-c cannot pass therethrough or thereover in the delivery system 200. The inner catheter 205 includes a tapered distal tip 235 and is moveable relative to the outer catheter 220. A stopper band 225 may be positioned at a distal end 220a of the outer catheter 220 and slideably disposed about the inner catheter 205. Like the outer catheter 220, the stopper band 225 is positioned proximal to the three stents 210a-c. The stopper band 225 is sized to contact a proximal end of the proximal-most stent 210c and is preferably bonded or otherwise secured (e.g., by heat or adhesive) to the distal end 220a of the outer catheter 220. A tubular sheath 230 may overlie the outer catheter 220 and the three stents 210a-c prior to deployment, leaving the distal tip 235 exposed.

The delivery system may also be configured to sequentially deploy more than three medical devices, such as, for example, four, five, or six devices. In each embodiment, the system includes separator bands between longitudinally adjacent devices, for a total number of separator bands equal to one fewer than the number of devices. For example, a delivery system including four medical devices would include three separator bands slideably disposed about an inner catheter. Preferably, the separator bands are radiopaque separator bands formed partly or entirely of a radiopaque material. Further, each embodiment of the system includes an outer catheter overlying the inner catheter and positioned proximal to the medical devices. The outer catheter is preferably sized so that the medical devices cannot pass therethrough or thereover, and may include a stopper band (e.g., a radiopaque stopper band) at its distal end. A tubular sheath may overlie the outer catheter, the bands, and the medical devices prior to deployment.

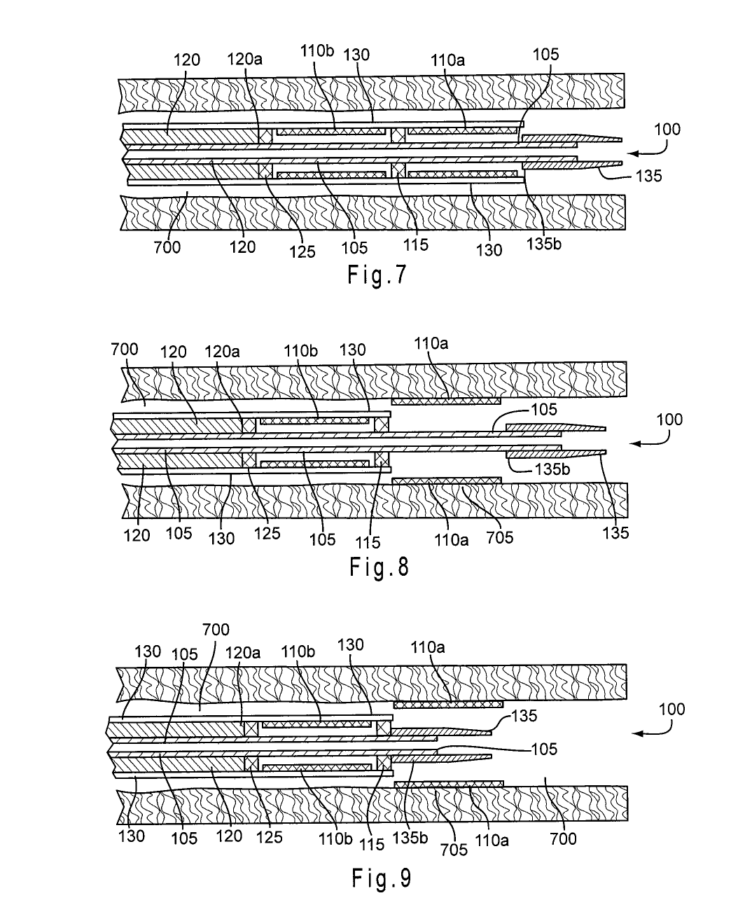

A method of sequentially deploying two or more medical devices using the improved delivery system is described in reference to FIGS. 7-11.

The method includes providing an intraluminal delivery system 100 having a delivery configuration including two or more implantable medical devices 110a, 110b arranged longitudinally adjacent to each other about an inner catheter 105. As described above, at least one separator band 115, which may be formed partly or entirely from a radiopaque material, is slideably disposed about the inner catheter 105 between longitudinally adjacent devices 110a, 110b. An outer catheter 120 is proximal to the implantable medical devices 110a, 110b and overlies the inner catheter 105, which is moveable relative to the outer catheter 120. A radiopaque stopper band 125 may be positioned at a distal end 120a of the outer catheter 120 and slideably disposed about the inner catheter 105. Like the separator band 115, the stopper band 125 may be made in whole or in part of a radiopaque material. A tubular sheath 130 may overlie the outer catheter 120, the bands 115, 125, and the implantable medical devices 110a, 110b.

Generally, the medical devices 110a, 110b are self-expanding medical devices. In FIGS. 7-11, the medical devices 110a, 110b are self-expanding stents. A first stent 110a resides in the delivery system distal to the second stent 110b. The distal-most (first) stent 110a is deployed prior to the second stent 110b at a first treatment site, and then the second stent is deployed at a second treatment site, which is either distal to, proximal to, or the same as the first treatment site. The second treatment site may be in the same or in a different body vessel from the first treatment site.

Referring to FIGS. 7 and 8, the method entails advancing the intraluminal delivery system within a body vessel 700 to a first treatment site 705, and deploying the first stent 110a at the first treatment site 705. To deploy the first stent 110a, the tubular sheath 130 and the inner catheter 105 are moved relative to each other. Generally, the inner catheter 105 remains in position at the first treatment site 705 while the tubular sheath 130 is retracted in a proximal direction. Once the first stent 110a is freed of the radial constraint provided by the overlying sheath 130, it is free to radially expand to contact the vessel wall. During deployment, the separator band 115 prevents contact between the first and second stents 110a, 110b, and the stopper band 125 and outer catheter 120 minimize proximal motion of the second stent 110b.

Referring to FIG. 9, after deploying the first stent 110a, the inner catheter 105 and the outer catheter 120 are moved relative to each other in order to position the distal tip 135 of the inner catheter 105 adjacent to the separator band 115. Generally, the inner catheter 105 is withdrawn relative to the outer catheter 120 to retract the distal tip 135, i.e., to move the distal tip 135 in a proximal direction. Having the distal tip 135 positioned adjacent to the separator band 115 and close to the second stent 110b allows for a more compact delivery system 100 that is less likely to cause vascular or organ damage downstream of the first treatment site 110a.

Having a more compact delivery system 100 may be particularly important if, after deploying the first stent 110a, the delivery system 100 is repositioned to a second treatment site which is distal to the first 110a. Without the ability to retract the distal tip 135 to a position close to the separator band 115 and the second stent 110b, the distal tip 135 could extend tens of millimeters beyond the second stent 110b (depending on the length of the first stent 110a). Such a configuration could inhibit deployment of the second stent 110b at the intended treatment site or cause unacceptable damage to downstream organs during repositioning of the delivery system 100.

It is also envisioned that the delivery system 100 may be repositioned to a second treatment site which is proximal to the first treatment site 110a after deploying the first stent 110a.

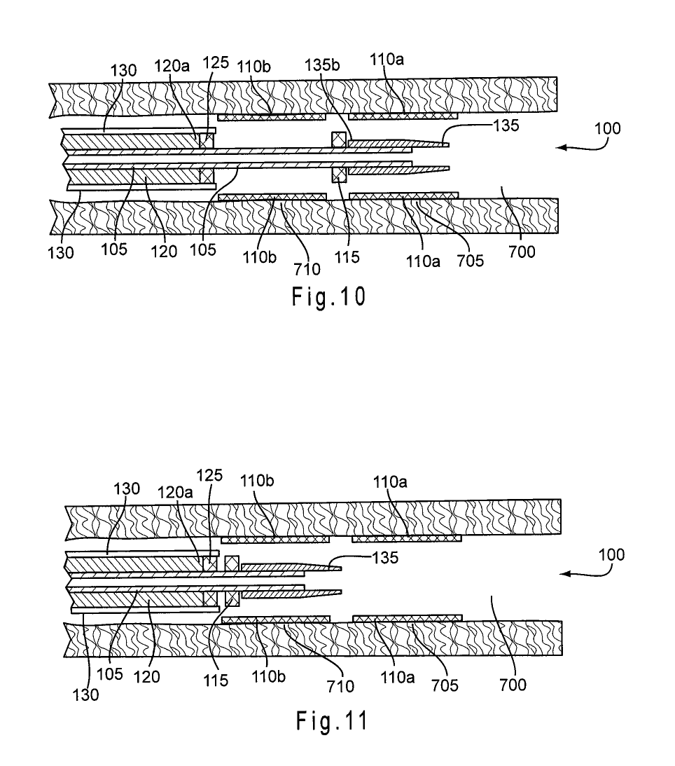

Or, referring to FIGS. 10 and 11, the second stent 110b may be deployed at a more proximal location without repositioning the delivery system 100. In FIGS. 10 and 11, the second treatment site 710 is immediately proximal to the first treatment site 705, and thus no movement of the inner catheter 105 is needed after deploying the first stent 110a in order to properly position the second stent 110b for deployment. It is also possible for the delivery system 100 to be moved a short distance in a distal direction after deploying the first stent 110a to allow the second stent 110b to be deployed at the same treatment site 705 as the first stent 110a.

In a situation in which the delivery system includes three or more implantable medical devices, as shown for example in FIG. 6, the three or more devices may be deployed at one or more treatment sites in the same body vessel or in different body vessels. For example, in the case of a delivery system having three medical devices (i.e., first, second and third medical devices 210a-c) to be deployed at respective first, second, and third treatment sites, the first treatment site may be either distal to or proximal to the second and third treatment sites. Alternatively, the first treatment site may be distal to one of the second and third treatment sites but proximal to the other one. It is also possible for the first treatment site to be the same as one or both of the second and third treatment sites.

After each of the first, second, and third medical devices is deployed, the inner catheter may be withdrawn to position the distal tip adjacent to one of the bands. For example, after the first medical device is deployed, the distal tip may be retracted to a position adjacent to the first (more distally positioned) separator band, and, after the second medical device is deployed, the distal tip may be further retracted to a position adjacent to the second (more proximally positioned) separator band. Finally, after the third device is deployed, the distal tip may be fully retracted to a position adjacent to the stopper band (or to the distal end of the outer catheter in an embodiment in which the stopper band is not used). The retraction of the distal tip all the way back to the stopper band or outer catheter may occur incrementally as described above, where the tip is partially retracted after each medical device is deployed, or the retraction may occur in a single motion only after all of the medical devices have been deployed.

The medical devices positioned about the inner catheter of the delivery system may have different expanded diameters and/or different lengths. For example, a first stent of a first expanded diameter and a first length may be deployed at a first treatment site that requires a stent of a given size, and a second stent of a second expanded diameter and a second length may be deployed at a second treatment site that requires a stent of a larger or smaller size. There is no particular limitation on the sizes of the intraluminal medical devices that may be employed in the improved delivery system. For example, stents ranging in length from about 10 mm to about 100 mm may be delivered and deployed, and the stents may have expanded diameters ranging from about 4 mm to about 12 mm.

Furthermore, the two or more medical devices to be deployed may be different devices. For example, a first of two devices may be an embolic protection filter and the second may be a self-expanding stent. In another example using three devices, the first device may be a nonhydrated tube of small intestinal submucosa (SIS), the second device may be a first self-expanding stent, and the third device may be a second self-expanding stent. In this case, the SIS tube may be delivered to a stenosed region and hydrated (and thus expanded), and the first stent may self-expand proximal of the stenosis to anchor the SIS tube in place. The distal tip of the inner catheter may be retracted to a position adjacent to one of the separator bands before and/or after deploying the first stent. The delivery system may then be advanced through the stenosis to place the second stent at the site of the hydrated SIS tube, and then the second stent may be expanded with sufficient radial force to compel the hydrated SIS tube against the vessel wall.

The separator and stopper bands may be made entirely or in part of a radiopaque material. Preferably, the radiopaque material is also biocompatible. A radiopaque material preferentially absorbs incident x-rays and tends to show high radiation contrast and good visibility in x-ray images. A material that is not radiopaque tends to transmit incident x-rays and may not be readily visible in x-ray images. Accordingly, the term “radiopaque material,” as used here, refers to a material that is substantially opaque to x-ray radiation and is thus readily visible using an x-ray imaging device or in an x-ray image.

The radiopaque material of the separator and/or stopper bands may be a radiopaque metal or alloy that includes, for example, one or more elements selected from the group consisting of gold, hafnium, iridium, niobium, osmium, palladium, platinum, rhenium, rhodium, ruthenium, silver, tantalum, and tungsten.

The bands may alternatively be formed of a polymer that includes radiopaque particles and/or compounds that raise the radiopacity of the polymer to a level sufficient for viewing by x-ray fluoroscopy. The term “radiopacity” refers to the capacity of a material or object to absorb incident electromagnetic radiation, in particular, x-ray radiation. Radiopaque polymers are described in, for example, U.S. Pat. No. 6,040,408, “Radiopaque polymers and methods for preparation thereof,” which issued on Mar. 21, 2000, and is hereby incorporated by reference in its entirety.

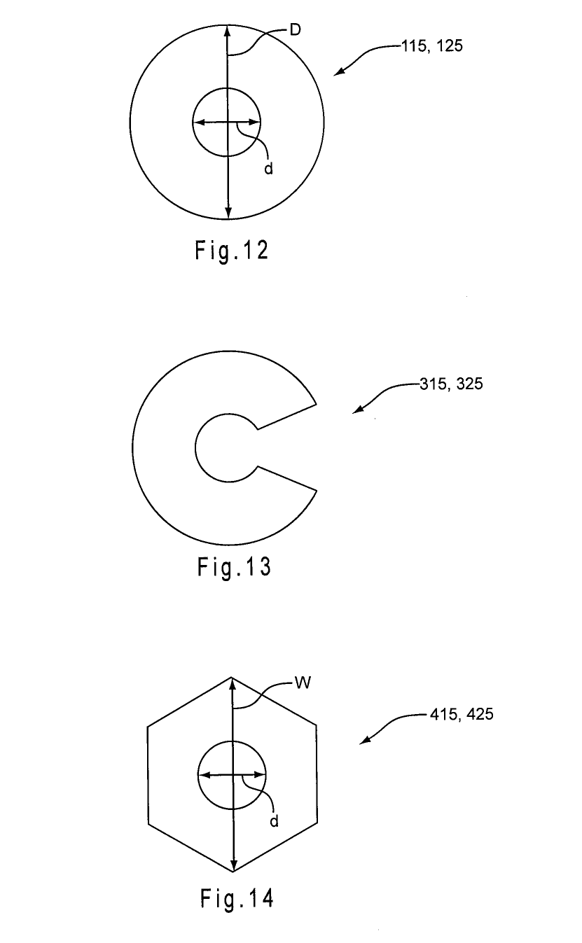

The stopper band and separator band(s) may have an annular (ring-like) shape and may extend entirely about the circumference of the inner catheter, as shown in the transverse cross-sectional view of an exemplary radiopaque band 115, 125 in FIG. 12. Alternatively, the bands 315, 325 may extend only partway about the circumference, having, for example, a C-shape, as shown in FIG. 13, or another circumferentially discontinuous configuration. The stopper band in particular may include multiple pieces or segments that are affixed to, embedded within, or otherwise secured to the outer catheter. Preferably, the pieces or segments are formed of a radiopaque material.

Each band may be cut from a metal or polymeric tube sized to fit over the inner catheter. Typically, the band has smoothly curved inner and outer surfaces, as shown in FIGS. 12 and 13. It may be advantageous in terms of x-ray opacity, however, for the band to have a faceted outer surface including a plurality of facets 405, as shown for example in FIG. 14. The exemplary stopper or separator band 415, 425 of this figure includes six facets, but other numbers are also possible. For example, the separator band may include four, five, seven, eight, nine, or ten facets on its outer surface. A faceted band may also have a circumferentially discontinuous structure.

Each separator band preferably has a longitudinal dimension or thickness sufficient to keep the medical devices separated from each other, even under loading from adjacent devices, and to permit viewing under x-ray irradiation. For example, the thickness is preferably at least about 0.5 mm. Generally, the thickness is no more than about 10 mm. For example, the thickness may lie in the range of from about 1 mm to about 5 mm. The stopper band may also have a thickness or longitudinal dimension in the range of from 0.5 mm to about 10 mm, or from about 1 mm to about 5 mm.

Referring to FIGS. 3 and 12, the separator band 115 has an inner diameter d sufficiently large to allow for motion along the inner catheter 105, but small enough that the band 115 cannot be dislodged from the delivery system 100 by passing over the tapered distal tip 135. Accordingly, the inner diameter d of the band 115 is less than an outer diameter of the distal tip 135. The inner diameter d of the separator band 125 is also small enough to prevent the adjacent medical devices 110a, 110b from passing therethrough in the delivery system 100.

Referring to FIGS. 1, 12, and 14, the separator band 115 has an outer diameter D (or width W) that is small enough to be contained within an overlying tubular sheath 130 but large enough to make contact with the thin-walled medical devices 110a, 110b that lie adjacent to the separator band 115 and press radially outward against the sheath 130 during delivery into the body vessel. Accordingly, the outer diameter D of the band 115 may be less than the inner diameter of the tubular sheath 130 but greater than the inner diameter of the sheath 130 minus two times the wall thickness of the implantable medical devices 110a, 110b (or, in the case of devices having different wall thicknesses, the wall thickness of the thinnest-walled medical device). For example, the separator bands 115 may have an inner diameter in the range of from about 0.5 mm to about 2 mm, and an outer diameter in the range of from about 1 mm to about 3 mm.

Similarly, referring to FIG. 1, each of the outer catheter 120 and the stopper band 125 has an inner diameter large enough to allow the underlying inner catheter 105 to be advanced and retracted, but preferably small enough to prevent the adjacent medical device 110b from passing therethrough. Additionally, an outer diameter of the stopper band 125 and/or the outer catheter 120 is large enough to make contact with the adjacent thin-walled medical device 110b that presses radially outward against the tubular sheath 130 during delivery into the body vessel. As with the separator bands 115, the outer catheter 120 and/or the stopper band 125 preferably has an outer diameter which is less than the inner diameter of the sheath 130 but greater than the inner diameter of the sheath 130 minus two times the wall thickness of the adjacent implantable medical device 110b. In other words, the outer diameter one or both of the outer catheter 120 and stopper band 125 is larger than an inner diameter of the stent 110b when compressed in the delivery system 100. For example, the outer catheter 120 and stopper band 125 may have an inner diameter in the range of from about 0.5 mm to about 2 mm, and an outer diameter of in the range of from about 1 mm to about 3 mm. The outer catheter 120 and the stopper band 125 need not have the same radial dimensions.

The outer and inner catheters 120, 105, tubular sheath 130, and distal tip 135 of the improved delivery system may be fabricated from tubing extruded from one or more biocompatible polymers. For example, the polymer(s) may include one or more of polyamide (e.g., nylon), thermoplastic fluorocarbon (e.g., fluoroethylene-propylene (FEP)), polyether block amide (PEBA), polyolefin, polyimide, polyurethane, polyvinyl chloride (PVC), and PEEK™. The sheath or catheters may be reinforced with a metal or alloy wire, cable or mesh to improve kink resistance and pushability. The tubing employed to fabricate the distal tip of the inner catheter may undergo an elevated temperature forming operation to achieve the desired tapered shape of the tip.

An improved intraluminal delivery system and a method for sequentially deploying two or more implantable medical devices have been described. The system and method permit two or more medical devices to be deployed at multiple locations in one or more body vessels in a single procedure. The first device may be deployed at a treatment site that is distal to, proximal to, or the same as that of a succeeding device. The system includes a retractable distal tip that allows the delivery system to be made more compact in vivo as the medical devices are deployed. Accordingly, the system and method may provide increased flexibility in the placement of the implantable medical devices while reducing the likelihood of vascular or organ damage downstream of the treatment sites. The system is also designed to minimize contact between and potential damage to adjacent medical devices during deployment.

Although the present invention has been described in considerable detail with reference to certain embodiments thereof, other embodiments are possible without departing from the present invention. The spirit and scope of the appended claims should not be limited, therefore, to the description of the preferred embodiments contained herein. All embodiments that come within the meaning of the claims, either literally or by equivalence, are intended to be embraced therein. Furthermore, the advantages described above are not necessarily the only advantages of the invention, and it is not necessarily expected that all of the described advantages will be achieved with every embodiment of the invention.

1. A method for sequentially deploying two or more implantable medical devices, the method comprising:
providing an intraluminal delivery system having a delivery configuration comprising:
two or more implantable medical devices arranged longitudinally adjacent to each other about an inner catheter;
one or more separator bands slideably disposed about the inner catheter, each separator band positioned between longitudinally adjacent medical devices;
an outer catheter overlying the inner catheter and disposed proximal to the medical devices, the outer catheter having an inner diameter large enough to allow longitudinal motion of the inner catheter but small enough to prevent the medical devices from passing therethrough; and
advancing the intraluminal delivery system to a first treatment site in a body vessel;
deploying a first medical device at the first treatment site;
deploying a second medical device at a second treatment site;
moving the inner catheter relative to the outer catheter to retract a distal tip of the inner catheter after deploying at least one of the first and second medical devices; and
removing the intraluminal delivery system from the body vessel, the first and second medical devices remaining deployed.

2. The method of claim 1, wherein a tubular sheath overlies the outer catheter, the one or more separator bands, and the implantable medical devices, and wherein deploying the first medical device and deploying the second medical device comprise moving the tubular sheath and the inner catheter relative to each other.

3. The method of claim 2, wherein moving the tubular sheath and the inner catheter relative to each other comprises retracting the tubular sheath in a proximal direction.

4. The method of claim 1, wherein, after deploying the first medical device, the distal tip of the inner catheter is retracted to a position adjacent to the separator band.

5. The method of claim 1, wherein, after deploying the second medical device, the distal tip of the inner catheter and the separator band are retracted to a position adjacent to a distal end of the outer catheter.

6. The method of claim 1, further comprising, after deploying the first medical device, advancing the intraluminal delivery system to the second treatment site, the second treatment site being distal to the first treatment site.

7. The method of claim 1, further comprising, after deploying the first medical device, retracting the intraluminal delivery system to the second treatment site, the second treatment site being proximal to the first treatment site.

8. The method of claim 1, wherein the first treatment site and the second treatment site are the same.

9. The method of claim 1, further comprising, after deploying the second medical device, deploying a third medical device at a third treatment site.

10. The method of claim 9, further comprising, after deploying the third medical device, moving the inner catheter relative to the outer catheter to retract the distal tip of the inner catheter.

11. The method of claim 1, wherein a tubular sheath overlies the outer catheter, the one or more separator bands, and the medical devices, and wherein deploying the first medical device and deploying the second medical device comprise retracting the tubular sheath in a proximal direction;
wherein, after deploying the first medical device, the distal tip of the inner catheter is retracted to a position adjacent to the separator band;
wherein, after deploying the second medical device, the distal tip of the inner catheter and the separator band are retracted to a position adjacent to a distal end of the outer catheter, and
wherein a stopper band is disposed at the distal end of the outer catheter, the stopper band and the one or more separator bands comprising a radiopaque material.

12. An intraluminal delivery system for the sequential deployment of two or more implantable medical devices, the system having a delivery configuration comprising:
two or more implantable medical devices arranged longitudinally adjacent to each other about an inner catheter;
one or more separator bands slideably disposed about the inner catheter, each separator band positioned between longitudinally adjacent medical devices;
an outer catheter proximal to the medical devices and overlying the inner catheter but not overlying the medical devices; and
a tubular sheath overlying the outer catheter, the one or more separator bands, and the medical devices;
wherein relative motion between the tubular sheath and the inner catheter allows the medical devices to be sequentially deployed at one or more treatment sites, and wherein relative motion between the inner catheter and the outer catheter allows a distal tip of the inner catheter to be retracted after deployment of each device.

13. The intraluminal delivery system of claim 12, wherein the separator band comprises a radiopaque material.

14. The intraluminal delivery system of claim 12, wherein the separator band extends entirely about a circumference of the inner catheter.

15. The intraluminal delivery system of claim 12, wherein the separator band comprises a longitudinal dimension of from about 1 mm to about 5 mm.

16. The intraluminal delivery system of claim 12, further comprising a stopper band comprising a radiopaque material at a distal end of the outer catheter.

17. The intraluminal delivery system of claim 16, wherein the stopper band is secured to the distal end of the outer catheter.

18. The intraluminal delivery system of claim 16, wherein the stopper band is integrally formed with the distal end of the outer catheter.

19. The intraluminal delivery system of claim 12, wherein the two or more implantable medical devices are self-expanding devices of different expanded diameters.

20. The intraluminal delivery system of claim 12, wherein the two or more implantable medical devices are self-expanding stents.
